(19) United States
(12) Patent Application Publication
(10) Pub. No.: US 2022/0214328 A1
(43) Pub. Date: Jul. 7, 2022

(54) SYSTEM AND METHODS FOR DETECTION OF VOLATILE ORGANIC COMPOUNDS IN AIR

(71) Applicant: Koniku, Inc., San Rafael, CA (US)

(72) Inventors: Oshiorenoya E. Agabi, San Rafael, CA (US); Renaud Renault, San Rafael, CA (US)

(21) Appl. No.: 17/571,363

(22) Filed: Jan. 7, 2022

Related U.S. Application Data
(60) Provisional application No. 63/134,830, filed on Jan. 7, 2021.

(51) Int. Cl. G01N 33/50; B01L 3/00

(52) U.S. Cl. CPC G01N 33/5005; B01L 3/502715; B01L 2300/0887; B01L 2300/10; B01L 2300/0883; B01L 2300/168; B01L 2300/0819; B01L 2300/044

(57) ABSTRACT

A biochip for detection of volatile organic compounds in air includes one or more wells for holding living cells. A capillary connecting each well to a liquid source may be used. The liquid source may be an on-chip reservoir or a system liquid supply. An air flow channel is separated from each well by a membrane. At least a portion of the biochip is transparent to allow optical detection of cell fluorescence. The biochip may be made of multiple flat transparent layers attached together. A system for detecting volatile organic compounds in air has an optical system adapted to detect fluorescence of genetically modified living cells expressing an odorant receptor capable of binding to the volatile organic compound and a calcium sensitive fluorescent reporter that fluoresces in response to binding of the volatile organic compound to the odorant receptor.

This application claims priority to U.S. Patent Application No. 63/134,380 filed Jan. 7, 2021, now pending and incorporated herein by reference.

The field of the invention is detection of volatile organic compounds (VOCs) in the air.

### BACKGROUND OF THE INVENTION

Volatile organic compounds (VOCs) are natural or manmade compounds which readily diffuse into air, due to their volatile characteristics. Many VOCs are toxic to humans and the environment with extended exposure. VOCs are also associated with explosives. Thus, detecting VOCs is important to human safety and security, and for better preserving the environment. Although various techniques have been proposed and used for detecting VOCs, they have been met with only varying degrees of success. Accordingly, improved systems and methods for detecting VOCs are needed.

### Overview

A system for detecting VOC's uses living biological cells. From an evolutionary perspective, biological cells as a system have been fine-tuned over millions of years for the purpose of sensing various molecules. Cells have evolved to be energetically efficient and sturdy. Cells can repair themselves and adapt to environmental changes. Cells can also be reprogrammed and manipulated in a variety of ways through genetic modifications.

In humans, the sense of smell is generally achieved by a type of neuron located in the nasal epithelium, which express olfactory or odorant receptors (OR) on their surfaces. Each odorant neuron usually expresses only one OR gene among the hundreds present in the organism's genome. When an odorant molecule, or VOC, from inhaled air binds to a matching receptor, the event triggers a chain of reactions that result in electrical signals. These signals, or spikes, propagate into the brain and are further processed to give rise to a complex sense of smell.

A cell may be modified to express a receptor. The receptor may be an odorant receptor. The receptor may be a wild-type receptor. The receptor may be a modified receptor, such as a genetically modified receptor. A receptor may be modified to enhance a binding specificity to a particular compound or to alter the receptor from a broadly tuned receptor to a narrowly tuned receptor or vice versus. The cell may be modified to express only one unique receptor, or more than one unique receptor. The cell may be modified to express two unique receptors. The cell may be modified to express three or more unique receptors. A receptor may be a human receptor, a mouse receptor, a canine receptor, an insect receptor, or other species type of odorant receptor.

OR activation eventually results in an increase in cytosolic calcium concentration, which can be measured using a calcium sensitive fluorescent reporter. These may include FIP-CBSM, Pericams, GCaMPs TN-L15, TNhumTnC, TN-XL, TN-XXL, Twitch's, RCaMP1, jRGECO1a, or any other suitable genetically encoded calcium indicator. The binding of an odorant molecule to its receptor induces an increase in the fluorescence emitted by the cells. An optical detector can therefore be used to measure cellular response in a contactless manner. The present system and methods detect VOC's using an optical detector that detects fluorescence.

A biochip used in the present system has one or more wells containing genetically modified living cells expressing an odorant receptor capable of binding to a volatile organic compound, and a fluorescent reporter that fluoreses in response to binding of the volatile organic compound to the odorant receptor. A capillary connects each well to a liquid source. An air flow channel is separated from each well by a membrane. Living cells are bound to a first side of the membrane, and a wall of the air flow channel is formed by a second side of the membrane. At least a portion of the biochip may be transparent.

Other objects, features and advantages will become apparent from the following detailed description and drawings, which are provided as examples for explanation, and are not intended to be limits on the scope of the invention.

### BRIEF DESCRIPTION OF THE DRAWINGS

In the drawings, the same element number indicates the same element in each of the views.

FIG. 1 is a schematic diagram of a VOC detection system.

FIG. 2 is a schematic diagram of the optical system of the VOC detection system of FIG. 1.

FIG. 3 is a bottom perspective view of a microfluidic biochip.

FIG. 4 is a top perspective view of the microfluidic biochip shown in FIG. 3.

FIG. 5A is a bottom perspective view of the microfluidic biochip of FIGS. 3 and 4 with the top foil or seal layer shown in FIG. 4 removed for purpose of illustration.

FIG. 5B is a bottom perspective view of an alternative microfluidic biochip with the top foil or seal layer removed for purpose of illustration.

FIG. 5C is a bottom perspective view of another alternative microfluidic biochip with the top foil or seal layer removed for purpose of illustration.

FIG. 6 is an exploded top perspective view of the microfluidic biochip shown in FIGS. 3 and 4.

FIG. 7 is a schematic representation of an osmolarity control system.

FIG. 8 is a front perspective view of a detection system with the top cover removed for purpose of illustration.

FIG. 9A is a side perspective view of the detection system of FIG. 8 with the top cover in place.

FIG. 9B is a side perspective view of the detection system of FIG. 8 having an alternative water collection container on the outside of the cover.

FIG. 10 is an enlarged front view of components of the detection system shown in FIGS. 8 and 9.

FIG. 11 is a front view of components of the detection system shown removed from the housing.

FIG. 12 is a top view of the optical system having four optical channels, for use in the detection system shown in FIG. 1.

FIG. 13 is a front view of a biochip loader.

FIG. 14 is a side view of the biochip loader shown in FIG. 13.

FIG. 15 is a top view of the biochip loader shown in FIGS. 13 and 14.

FIG. 16 is a top view of the biochip loader of FIGS. 13-15 positioned for loading and unloading biochips from the detection system shown in FIGS. 8-12.

### DETAILED DESCRIPTION

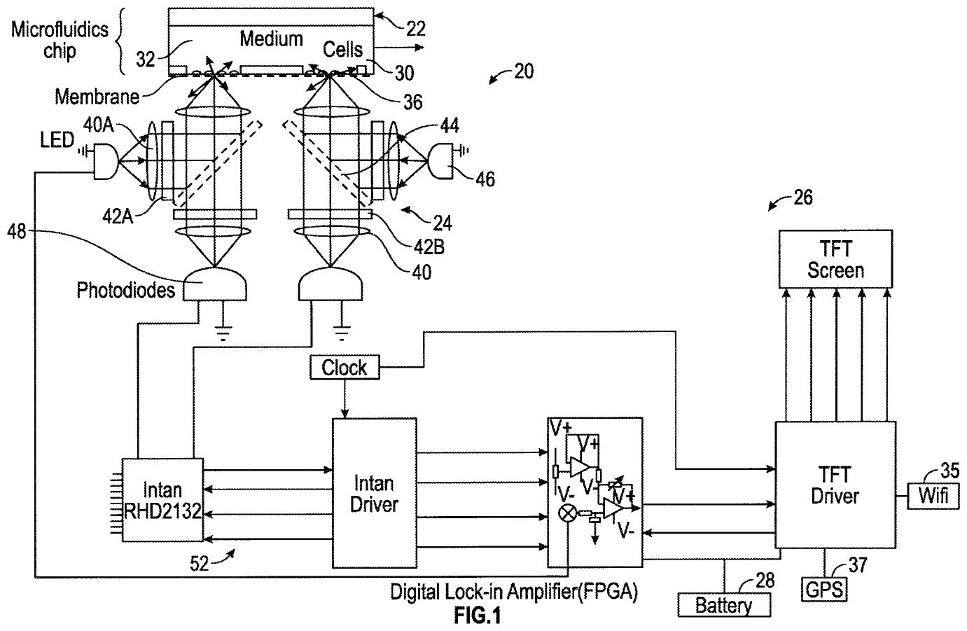

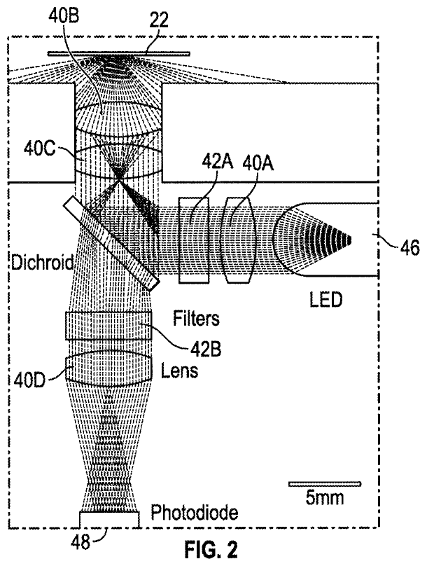

Referring to FIGS. 1 and 2, in a basic form, a VOC detection system 20 includes a cell carrier or substrate, such as a microfluidic biochip 22, an optical system 24 and an electronic system 26. The microfluidic biochip 22 contains cells 30, medium or water 32, and a membrane 36 which provides a barrier for the cells against contaminants such as viruses, bacteria and dust. The cells bind to the membrane 36, allowing the cells to more effectively interact with airborne odorants such as VOC's. Each channel or optical pathway of the optical system 24 includes one or more: light emitter, such as a blue LED 46, lenses 40A, 40B 40C and 40D, optical filters 42A and 42B, dichroic mirror 44, and a photodetector such as a photodiode 48.

FIG. 1 shows an embodiment having two optical pathways each having the above-listed elements, although the system may be designed with a single optical pathway or multiple optical pathways, depending on the intended application. The electronic system 26 in FIG. 1 is electrically connected to the blue LEDs 46 and to the photodiodes 48 and may include a digital lock-in amplifier 52 in the form of a field programmable gate array (FPGA). The electronic system 26 has an output device, such as a thin film transistor (TFT) display. Alternatively, the output or reporting from the detection system 20 may be provided via a WIFI, cellular, RF or wired connection. The electronic system 26 may include a GPS unit for detecting and reporting the location of the detection system 20. The electronic system 26 may also include control software or circuitry, and memory for recording detection events and other data. The detection system 20 may be powered by a battery 28, to allow flexibility in placement and use.

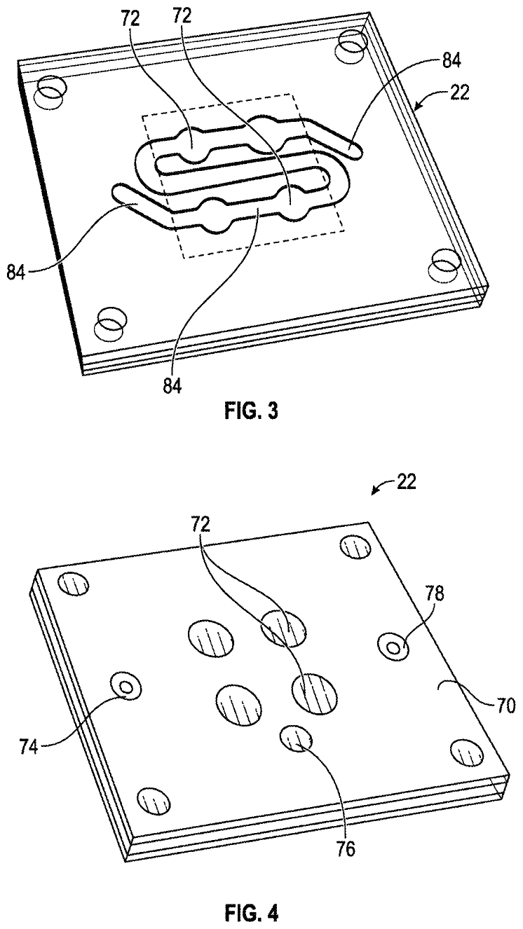

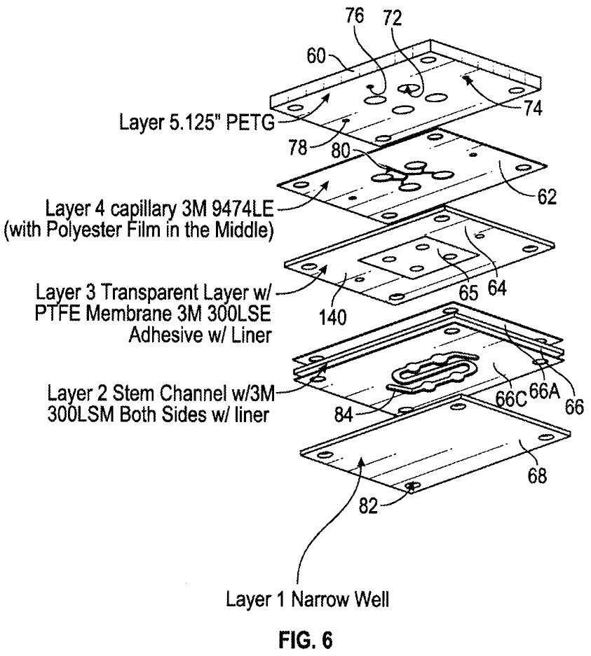

Turning now to FIGS. 3-6, in the example shown, specifically in FIG. 6, the microfluidic biochip 22 has a bottom or first layer 68, intermediate layers including a second layer 66, a third layer 64, and a fourth layer 62, and a fifth or top layer 60. The layers may be laser cut from PET plastic sheets (polyethylene terephthalate) or other materials, such as silicon, fused-silica, glass, any of a variety of polymers, e.g., polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), epoxy resins, metals (e.g., aluminum, stainless steel, copper, nickel, chromium, and titanium), or any combination of these materials.

The layers may be attached and sealed together via an adhesive, solvent welding, clamping or by using bio-compatible double sided tape and a hot press. The layers may optionally be made of glass and/or PDMS (silicon-based organic polymer) assembled using plasma bonding. The layers below the cells are translucent or transparent, so that the cells may be exposed to a light source such as the blue LED 46, and so that fluorescence emitted by the cells may be detected by the photodiodes 48. The layers above the cells 30 may optionally be transparent so the cells may be viewed from above. If not, the layers above the cells may be an opaque material such as plastic or metal.

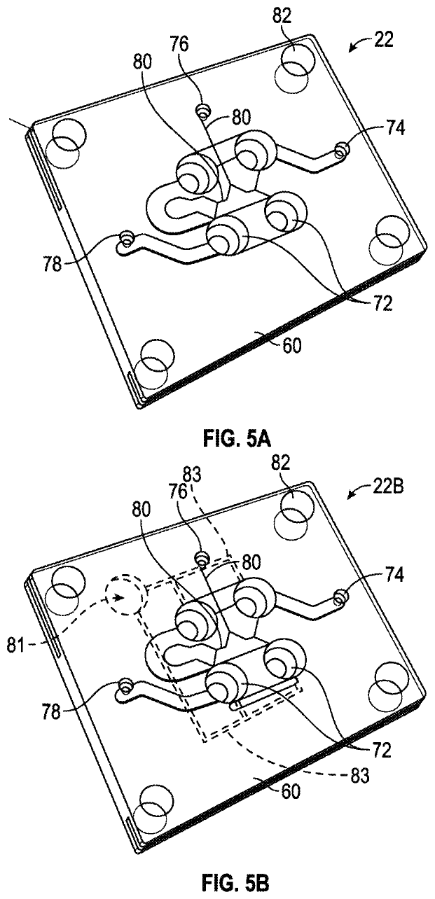

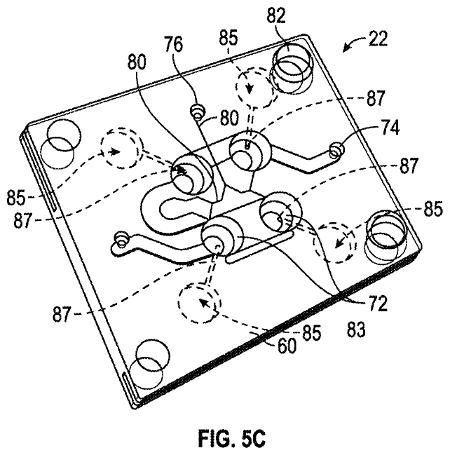

As shown in FIGS. 5 and 6, the fifth layer 60 and the fourth layer 62 have through holes providing wells 72 for holding cells 30. A membrane 65, such as a PTFE membrane, on the bottom surface of the third layer 64 closes off the bottom of the wells 72. The membrane may be treated to make it transparent and to promote cell adhesion. Cell adhesion to the membrane allows for better detection of VOCs, which move from the air flow channel 84 in the biochip 22 through the membrane 65. Although the example shown has four wells 72 in a square array, other numbers, patterns and shapes of wells may be used. Capillaries 80 in the fourth layer 62 connects a water inlet 76 in the fifth layer 60 into each of the wells 72. The capillaries 80 may be etched into the fourth layer 62 before assembly.

An air inlet 74 extends through the fifth layer 60, the fourth layer 62, the third layer 64 and connects into the air flow channel 84 which is formed in the second layer 66. As shown in FIG. 6 the air flow channel 84 extends under each of the wells 72, in an S-shaped configuration. The membrane 65 encloses the air flow channel 84 from above while the first layer 68 encloses the air flow channel 84 from below. The membrane 65 separates the cells 30 in the wells 72 from the air flow channel 84. The air flow channel 84 may be wider at positions under the wells 72, so that the cells 30 are better exposed to elements such as VOCs moving through the membrane 65. Alternatively, positions may be inverted with the air inlet 73 extending through the first or bottom layer. Where the biochip has an on-chip reservoir or liquid source as in FIGS. 5B and 5C, locating openings on the top of the biochip may be convenient for filling the reservoir(s) and/or the wells.

FIG. 6 shows the second layer 66 attached to the first layer 68 and to the third layer 64 using layers of double-sided tape 66A and 66C, as one example. The layers may alternatively be attached using adhesives, fasteners, plastics welding, or other techniques. Alignment holes 82 may be provided at the corners of each layer to precisely align the layers on a fixture during assembly of the layers into the microfluidic biochip 22.

FIGS. 5B and 5C show alternative biochips 22B and 22C which, unlike the biochip 22 in FIG. 5A, has no water inlet 76. Rather, the biochip 22b has an on-chip reservoir 81 filled with water or media. Water is supplied to each of the wells 72 via capillaries 83. The water may introduced into the biochip 22B when the cells 30 are placed into the wells. The water may contain nutrients. In FIG. 5C the biochip 22C has multiple separate reservoirs 85. Each reservoir 85 supplies water via a capillary 87 to a single well 72. Depending on the specific biochip design and number of wells, a single reservoir may supply water to all of the wells, as in FIG. 5B, or each well may be connected to a separate reservoir as in FIG. 5C, or one or more reservoirs may be connected to one or more wells. Biochips 22 having varying numbers of wells may be used, for example 2, 4, 8, 16, 32, 64, 96 or 98, 100, 128 and up to 1000 or above is specialized applications.

After the microfluidic biochip 22 is assembled and ready for use, cells 30 are placed into the wells 72 from the top of the fifth layer, the cells are seeded on top of the membrane 65, and the cells bind to the membrane 65. A foil or pierceable seal layer 70 may then be adhered onto the top surface of the fifth layer 60 to cover and seal the wells 72, as well as the water inlet 76, the air outlet 78, and the air inlet 74. The foil or seal layer 70 also prevents light from entering the top of biochip 22. This reduces evaporation and avoids stray light affecting the signal from the photodetectors. The microfluidic biochip 22 is then effectively sealed against the environment. The biochip 22 may be manufactured as a disposable unit intended for replacement e.g., every 30 days.

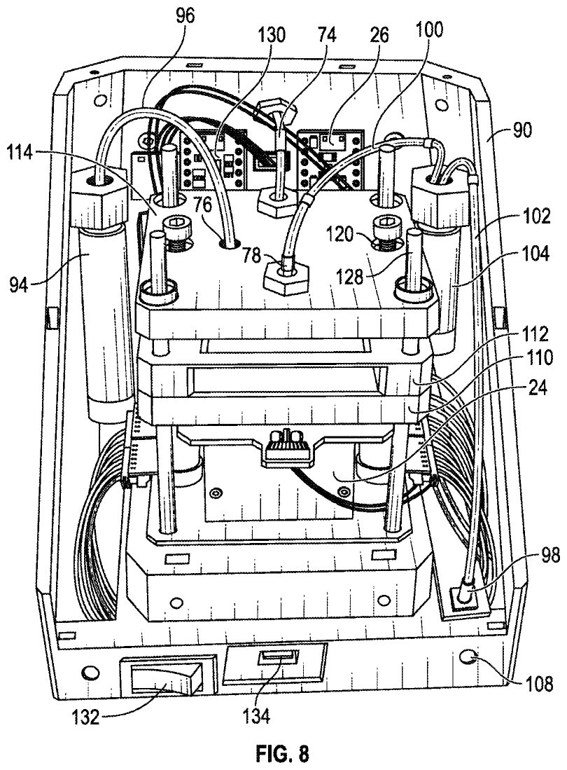

The microfluidic biochip 22 is designed for operation in the detection system 20 shown in FIGS. 1, 2 and 8-10, although it may also be used in other systems as well. Referring to FIGS. 8, 9 and 10, in the detection system 20, the optical system 24, the electronic system 26 and the battery 28 are contained within a housing 90. A frame 112 is positioned on top of the base 110. The frame 112 has a detection system slot or front opening 136 adapted to receive the microfluidic biochip 22. The base 110 and the frame 112 may be fixed in position on guide-posts 128. A top plate 114 is supported on one or more jack screws 120 which are rotated by one or more jack screw motors 122. The jack screws 120 and the jack screw motors 122 form an elevator to raise and lower the top plate 114 towards and away from the frame 112. Bushings at the corners of the top plate 114 slide on the guide-posts 128 and prevent lateral movement as the top plate 114 moves vertically. Alternatively, the top plate 114 may be fixed in position with the frame 112 and the microfluidic biochip 22 moved vertically.

A water or liquid medium supply container 94 is connected to a water supply tube 96 which passes through the top plate 114, at a position in alignment over the water inlet 76 of the microfluidic biochip 22, when the microfluidic biochip 22 is installed in the detection system slot 136. A vacuum tube 100 extends from a water collection container 104, through the top plate 114, to a position aligned over the air outlet 78 of the microfluidic biochip 22 when the microfluidic biochip 22 is installed in the detection system slot 136. An air inlet tube similarly extends through the top plate 114 to a position aligned over the air inlet 74 of the biochip 22. With the top plate 114 in the up position, the biochip is sealed from the environment. When the top plate 114 moves down to engage the biochip 22, the water supply tube 96, the vacuum tube 100 and the air inlet tube pierce through the seal layer 70 to make fluid connections with the biochip 22.

A pump tube 102 connects the inlet of a vacuum pump 98 to the water collection container 104. The outlet of the vacuum pump 98 leads to an outlet 108. In an alternate design, a positive pressure pump may be used instead of the vacuum pump 98, with air pumped into the air inlet and through the air flow channel under positive pressure, rather than drawing air through the air flow channel via vacuum.

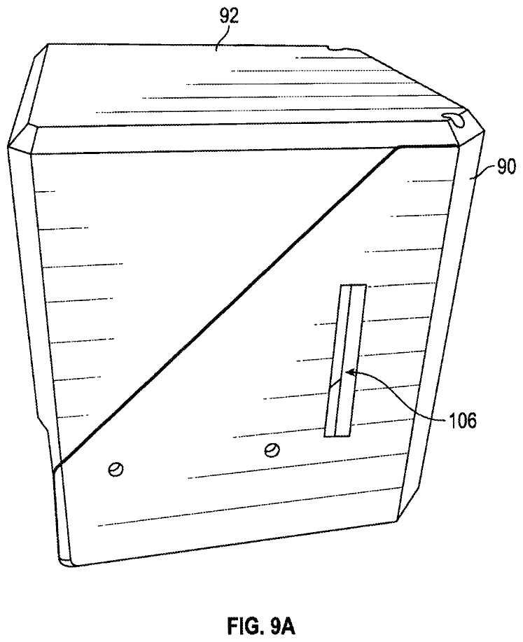

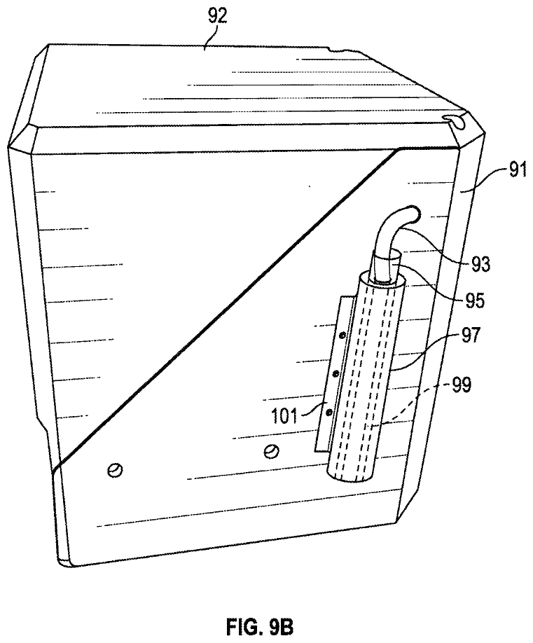

The detection system components may be in or on a housing 90 enclosed by a cover 92. As shown in FIG. 9A, sight windows 106 may be provided through side walls of the housing 90 aligned with the water supply container 94 and the water collection container 104, to allow visual inspection of the water level in the containers. The detection system 20 does not actively remove water from the biochip 22. However, humidity in the air moving through the air flow channel may condense into liquid water, which moves into and is collected in the water collection container 104.

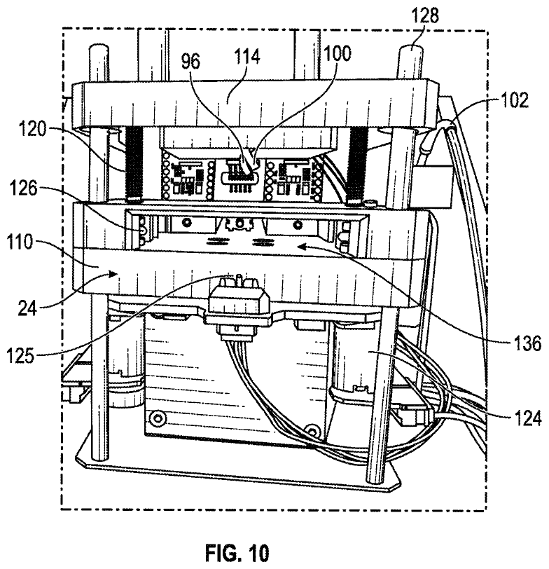

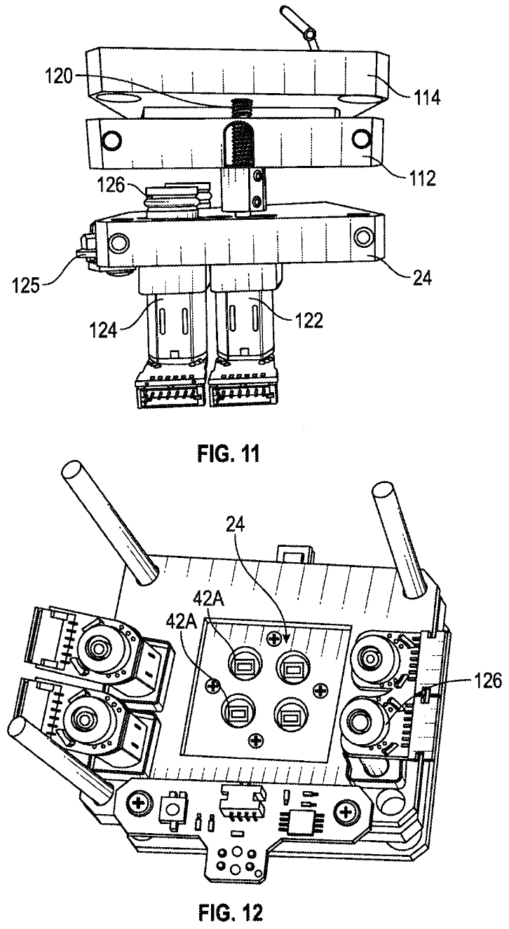

As shown in FIG. 8, the outlet 108 may extend through a front wall of the housing. Also as shown in FIG. 8, the electronic system 26 may include an on/off switch 132 on the housing 90, and a USB port 134 for charging the battery 28 or for interfacing the electronic system 26 to another device via a USB cable. As shown in FIGS. 10-12, rollers 126 projecting into the detection system slot 136 are rotatable to guide the biochip 22 into the detection system slot 136. Optionally, the rollers 126 may be rotated by one or more load motors 124 for this purpose. In this case, one or more sensors or switches 125 detects the presence of the biochip at detection system slot, causing the load motors 124 to turn on. The load motors 124 and rollers 126 provide a biochip mover for moving the biochip 22 horizontally. Alternate forms of biochip movers may be used instead of the load motors 124 and rollers 126, such as linear actuators, rack and pinion platforms, solenoids, etc. A biochip mover may be provided with single direction actuators and/or spring elements. The battery 28, the LEDs 46 and photodiodes 48 of the optical system, the jack screw motor 122 and the load motor 124 are electrically connected to a control board 130 of the electronic system 26, which controls the operations described below.

In use, cells 30 and water or medium are provided into the wells 72 of the microfluidic biochip 22. The foil layer 70 is then applied over the fifth layer 60 to seal the wells 72. The microfluidic biochip 22 is then ready for use, although the microfluidic biochip 22 may optionally also be stored for days or weeks with the cells having sufficient water and nutrients to maintain life.

The detection system 20 is placed in the desired location. Since the detection system is compact and requires no external connections, the detection system may be used in wide variety of locations. The detection system 20 is turned on via the switch 132. The microfluidic biochip 22 is loaded into the detection system slot 136. The jack screw motor 122 is turned on, rotating the jack screws 120 which lowers the top plate 114 towards the microfluidic biochip 22. The tips of the water supply tube 96 and the vacuum tube 100 pierce through the foil layer 70 and engage into the water inlet 76 and the air outlet 78 of the microfluidic biochip 22, respectively. The vacuum pump 98 is turned on, drawing air through the air flow channel 84. The optical system 24 is also turned on. An extension tube may optionally be provided on the air inlet to better sample air from a specific location rather than sampling ambient air around the detection system. In use, the air inlet or extension tube draws in an air sample or a volume of ambient air for testing for the presence of VOC's.

VOC's in the air drawn into the microfluidic biochip 22 pass through the membrane 65 and bind to an appropriate OR of the cells 30, transducing a signal that ultimately produces fluorescence when illuminated by the blue LED 46 or other light source reflected into the wells 72 by the mirror 44. When present, the fluorescence is detected by the photodiode 48. The detection event may then be displayed, transmitted and/or recorded.

With the tip of the water supply tube 96 engaged into the water inlet 76, water or other medium flows via capillary action from the water supply container 94 (if used) through the capillaries 80 and into the wells 72 to supply the cells 30. The cells 30 are consequently supplied with water from the capillaries 80 (via the water supply container or via an on-chip reservoir), and are exposed to VOC's passing through the membrane 65, but the cells 30 are otherwise sealed off from the environment.

When air sampling is completed, the microfluidic biochip 22 is removed or ejected from the detection system 20 and may be replaced with a new microfluidic biochip 22.

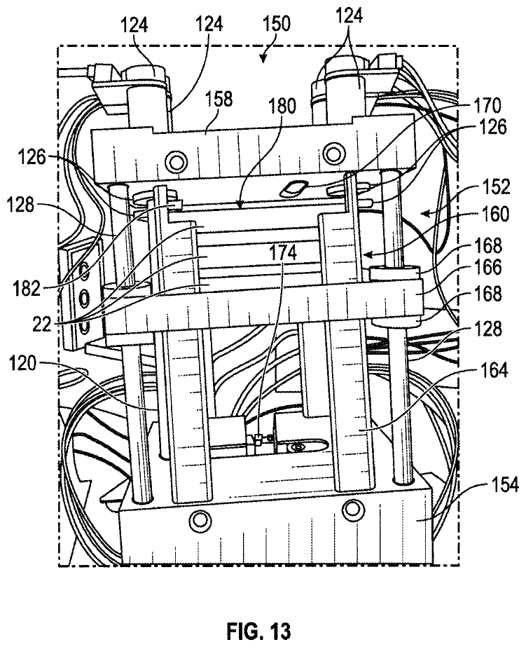

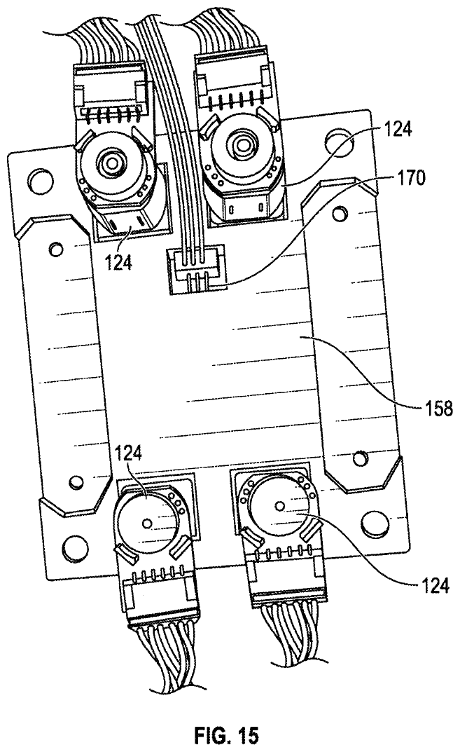

The detection system 20 may be provided with a biochip loader 150, together forming a combined unit 148, shown in FIG. 16, which can store multiple biochips 22 and automatically load and unload biochips 22 into and out of the detection system 20. The loader 150 allows the detection system 20 to operate unattended for an extended period of time. FIGS. 13-15 show the loader 150 with no housing. Generally, the loader 150 is contained within a housing which may be similar to the housing 90 shown in FIGS. 8-9. Alternatively, the loader 150 and the detection system 20 may be provided together in a single housing. In either case, the loader 150 is secured in a fixed position relative to the detection system 20, to allow biochips 22 to be moved between them. The loader 150 may also be electrically connected to the control board 130 or other component of the electronic system 26 of the detection system, with the control board 130 controlling both the detection system and the loader 150.

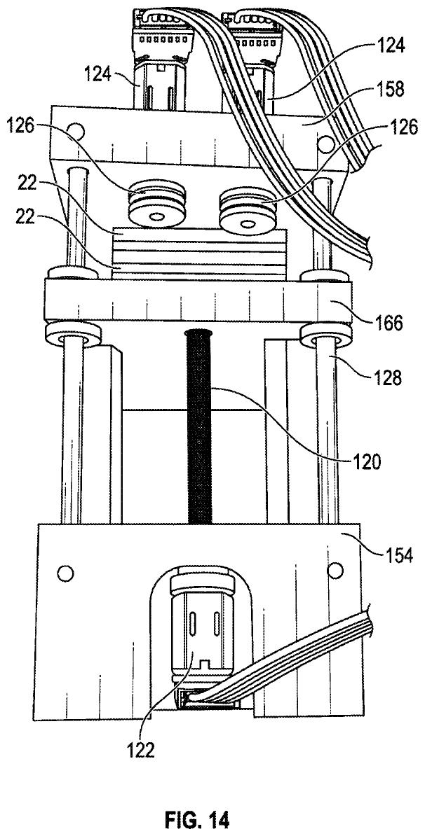

As shown in FIGS. 13-14, the loader 150 has a frame 152 including guide-posts 128 attached to a frame base 154 and a motor plate 158. A lift plate 166 is movable vertically on the guide-posts, driven by jack screw motors 122 rotating jack screws 120. Bushings 168 allow the lift plate 166 to slide vertically on the guide-posts 128 while reducing sliding friction and preventing lateral movement. A guideway 160 is formed within the frame 152 by columns 164 attached to the frame base 154 and the motor plate 158. The columns 164 pass through openings in the lift plate 166. The guideway 160 is configured to hold a stack of biochips 22 on the lift plate 166. A loader slot 180 is provided at the top of the guideway 160 to allow biochips 22 to be placed into the guideway 160.

FIG. 13 shows a stack of 3 biochips 22 in the loader 150, although the loader may have capacity to hold e.g., 2-10 or more biochips 22. FIG. 13 which is a front view of the loader 150, shows the loader slot 180 formed by openings or cut away sections 182 through the upper ends of the front columns 164. The rear columns may have the same design, so that the loader slot 180 extends entirely through the guideway 160 from the front to the back of the loader 150.

A limit switch or sensor 174 may be located at the bottom of the guideway 160 to sense when the lift plate 166 is in the full down position. A camera 170 or other optical detector may be provided on the bottom side of the motor plate 158 to visually detect the presence and/or number of biochips 22 in the loader 150, and/or to read an identifier on a biochip, such as a bar code on the seal layer. Referring to FIGS. 13-15, the loader 150 has a biochip mover, which may be provided in the forms of four load motors 124 on the motor plate 158. Each load motor rotates a roller 126, for moving biochips 22 into and out of the loader 150.

In use, the lift plate 166 of the loader 150 is lowered to or near the bottom of the guideway via the jack screw motors 122 rotating the jack screws 120. Multiple new or unused biochips 22 are inserted (by hand) through the loader slot 180 onto the lift plate 166 in the guideway 160. The biochips 22 may be keyed with the loader slot 180 so that the biochips can only be loaded in a single correct orientation. Alternatively, the biochips 22 may have a projection or other feature that allows loading in only the single correct orientation. In the combined unit 148, the detection system 20 and the loader 150 are fixed in position (e.g., bolted into place in a housing or a mounting plate), with the front of the loader 150 facing the front of the loader 150, and with the loader slot 180 of the loader adjacent to, and vertically and horizontally aligned with the detection system slot 136. In this design, the biochips 22 may be loaded into the loader 150 through the loader slot 180 at the back of the loader 150.

With the combined unit 148 placed or located in the desired room or space, the electrical system is turned on using the switch 132. The control board 130 confirms the presence of one or more biochips 22 in the loader 150, and optionally performs other functions, such as system checks, recording, reporting, etc. The control board activates the jack screw motors 122 to raise the lift plate 166 to vertically align the top-most biochip 22 with the loader slot 180. The load motors 124 of the loader 150 and the detection system 20 are turned on in the forward direction causing the rollers 126 to move the top-most biochip out of the loader 150 and into the detection system 20. The detection system 20 operates to detect VOC's as described above.

The cells in an operating biochip 22 can effectively operate for several days, for example from 3 to 10 days. The duration of biochip operation is a function of the ability of the receptors (OR) to last, and not of cell viability. Cells with improved ORs may be able to operate longer than 10 days. The ORs in cells in a sealed biochip may be stored in the loader 150 for up to six weeks. Regardless of the OR effective duration, after a prescribed time interval, or after other factors determine the ORs are no longer operating sufficiently, the control board 130 initiates replacement of the used biochip 22. The load motors 124 are turned in the reverse direction, with the load motors of the detection system 20 causing the used biochip 22 to move out of the detection system 20 and back into the empty loader slot 180 in the loader 150. The load motors 124 of the detection system 20, also rotating in the reverse direction, move the used biochip 22 through the empty loader slot 180 and the used biochip is ejected out from the back of the loader 150 into a collection location. The control board operates the jack screw motors 122 to lift the lift plate 166 to vertically align the next biochip in the guideway 160 with the loader slot 180. The load motors 124 are again switched on in the forward direction moving the next biochip from the loader 150 into the detection system 20. This sequence is continued until all of the biochips 22 in the loader 150 have been used. The control board may wirelessly communicate with a technician to provide detection results, and/or diagnostic and status data, or to allow the technician to remotely control operation of the combined unit 148.

When used with a biochip having water reservoir(s) as shown in FIGS. 5B and 5C, the water supply container 94 may be omitted. Referring to FIG. 9B, the water collection container 104 may be replaced by an external collection container 97 supported in a holder 101 on an outside surface of the cover 92. In this case the pump tube 102 is connected to the entry tube 93 of the external collection container 97, which may be removably secured into the external collection container 97 via a fitting 95. Water removed from the system is collected in the external collection container 97, which may contain a gel or other water absorbing material. The external collection container 97 may be removed and replaced with a new external collection container 97, without opening the cover, when the biochip is replaced, or after a selected number of biochips have cycled through the system.

The OR's (olfactory receptors) may be sequences extracted from the human (600 ORs) and mouse (1300 ORs) genomes, or from other animals such as dogs, elephants, insects, etc. Synthetic ORs with sequences that are not found in nature may be used. Such synthetic constructs are still considered ORs based on their sequence and functional similarities to natural ORs.

Cell types used include the Hana3A cell line, derived from the commonly used HEK293 (human embryonic kidney) cells. This cell line contains accessory proteins that help the expression of ORs, such as receptor transporter proteins RTP1, receptor expression enhancing proteins REEP1 and REEP2, as well as the protein Gaolf(s) necessary to transduce the signal. The second cell type which may be used is primary astrocytes, extracted from rat embryonic brains and expanded in vitro. Both cell types have been shown to function equally well in detecting VOCs. ORs as disclosed in U.S. Patent Application No. 63/189,015, incorporated by reference, may be used.

The number of cells needed to generate a measurable response depends on the brightness of the cells and the sensitivity of the fluorescence detector. In the portable system 20 described, about 10,000 cells are used for each well. In the design shown in FIG. 12, the optical system has four optical paths, one for each well, with each optical path including the components as shown in FIG. 2.

In the example shown in FIG. 1, band-pass filters 42 and a dichroic mirror 44 are used to separate excitation light from emitted light. The excitation source for each cell population may be a blue LED 46 (Nichia NSPB500AS) with a viewing angle of 15 degrees, coupled to a collimating lens (Thorlabs LB1157) and a blue excitation filter (Semrock FF01 469-35). The dichroic mirror (Semrock FF506) reflects the excitation light towards the cells in the biochip 22. A doublet of lenses focuses the excitation light onto the cells, and in turn collimates the emitted light back. Emitted light crosses the dichroic mirror and is filtered from scattered excitation light by a green emission filter (Semrock FF01 525-39). The filtered emitted light is focused by a lens on a silicon Photodiode 48 (Vishay VEMD5510C).

As shown in FIG. 2, the fluorescence reporter may be excited by blue light and emit green light. The conversion rate greatly increases (over 30 times) when the reporter is in the presence of calcium, which leads to an increase in emitted green light when the cells detect an odorant.

In order for the cells to generate a quick response, they are advantageously directly seeded on the membrane 65 that separates them from the outside environment. As it is difficult to embed electrodes on such thin membranes, the system monitors calcium flux in a contactless, optical way.

Fluorescence collected from one population depends on the number of cells and expression level of the calcium reporter protein. Cell number does not change where the system uses cells that do not divide such as neuronal cells. Cell number with cells that divide such as HANA3A cells, grow to a single layer confluence by dividing based on available space, and stop dividing when they touch each other. The number of functional fluorescent reporters in each cell can decrease over time due to natural protein turnover and photobleaching (light induced damage to fluorescent molecules). However, the cells may continuously produce new fluorescent proteins that compensate for this loss.

The fluorescence level is converted into a voltage by the photo-diode 48, and can easily be monitored or digitized for further processing. The change in fluorescence occurs at a timescale of a few seconds. At those low frequencies, the ambient electrical and optical noises affect the photo-diode voltage significantly more than the true fluorescence signal. This can be circumvented by providing the fluorescence signal a high frequency signature and filtering out the other frequencies. For example, the following steps may be used.

1. flashing the excitation LED 46 at 6 kHz, which causes in turn the fluorescence emission to have the same frequency.
2. multiplying the raw fluorescence signal with a reference signal of same frequency and same phase. Since the product of two periodic signals tends to zero when their frequencies are different, most of the noise (which isn't 6 kHz) is significantly attenuated.
3. smoothing the product with a low pass filter to remove the high frequency oscillations and only keep its DC component.

As shown in the example of FIG. 1, the initial analog to digital converter (ADC) step is performed by a low noise electrophysiology chip (Intan RHD2132) originally designed to record action potentials. The digital lock-in amplifier is designed in Verilog and implemented on a SPARTAN6 FPGA board. The lock-in output can be displayed on a TFT screen connected to the FPGA board, or sent to an on-board computer (Raspberry Pi Zero) through a custom parallel communication protocol.

An on-board computer can perform live analysis in order to translate the raw fluorescence intensity into detection events. This processing may consist first in computing the mean and standard deviation of the derivative of the signal over the previous 30 seconds. Detection occurs if the instantaneous derivative is greater than the average derivative+C times the RMS (dF>dF+C×((dF−dF)²))^{1/2} for at least n seconds, with and being chosen to favor either accuracy or speed of detection.

The membrane 65 on which the cells are living provides the interface that separates the controlled cellular environment from the outside air. The membrane may advantageously allow VOCs to diffuse across the membrane in seconds; prevent bio-contaminants from entering the cell medium and damaging the cells; be optically clear in order to visualize the cells; be chemically compatible with cell adhesion and growth; and be mechanically, chemically and heat resistant.

The membrane may be a thin (15 microns) PTFE (Teflon®) membrane with high porosity (75%) and a maximum pore size of 30 nm, which is smaller than bacteria and most relevant viruses. The membrane may be opaque when dry, however after wetting of the membrane with a low surface tension fluid like isopropyl alcohol, the membrane becomes transparent and can be kept transparent as long as one side is kept in contact with IPA, water, or cell medium. In spite of its thinness, the membrane is sturdy and can be heated to more than 200 degrees Celsius, which allows coating it with an anti-stiction material on the external side for some applications while improving cell adhesion by treating it with plasma, and incubating it with poly-D-lysine on the inward-facing side. A silicon oxide (SiO₂) membrane may also be used.

A pre-concentrator may be used to adsorb VOCs and desorb them upon heating.

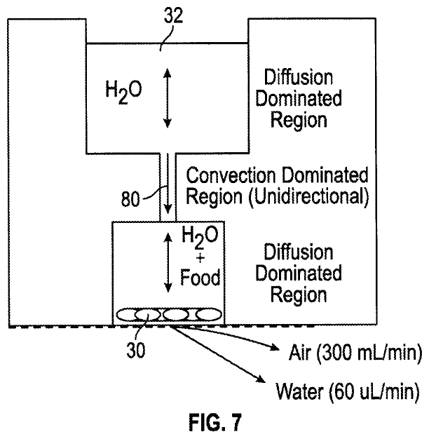

Evaporation of water or medium through the membrane into the air flow channel 84 is inherently tied to air sampling. Medium evaporation is one of the main causes of failure in cell culture. As water evaporates, the concentration of dissolved substances, such as salts, increase up to the point that the cells cannot function properly. Counter-acting this phenomenon helps to keep the cells alive. Referring to FIG. 7, measured rates of evaporation in the biochip of FIGS. 3-5 is on the order of 60 microliters per hour (40 mL/month for the biochip of FIGS. 3-5). This value is significant in comparison to the volume of medium that is sufficient for the cells to survive for one month. Indeed, based on the rate at which cells consume nutrients, they only require a few hundred microliters of medium per month. Perfusing fresh medium can compensate for this evaporation, but is wasteful since cells need water rather than fresh medium. However, perfusing pure water would flush away the vital solutes contained in the medium.

Thus the biochip is designed to use evaporation and capillary action inside the chip to aspirate water from a water supply container 94 or from the reservoir. If the water supply container is connected to the wells by thin enough capillaries 80, the speed of incoming water prevents solutes in the wells 72 from diffusing back into the water supply container, which insures that osmolarity remains constant inside the wells. The system also has the advantage to be self-regulated in a passive way. If the evaporation rate increases, the depression in the well will increase and draw water faster.

The vacuum pump 98 is driven by an electric motor which may use less than 0.5 W when on. There is no pump for water, as the transpirative osmolarity control system is passive. The vacuum pump 98 may run continuously, or intermittently, depending on the condition of the ORs and the status of the detection system.

As the example of FIGS. 3-5 uses mammalian cells, the optimal temperature is 37° C. Temperature control may be achieved by a single peltier module 140, attached to small aluminum overlay that distributes the heat over the four wells. The peltier element acts as a heat pump, transferring heat from one side of the unit to the other based on the direction of current flow through the device. An H-bridge circuit (DRV8838) may be used to control the current direction to either heat or cool the wells based on the temperature measured with an internal thermocouple (MAX31855). The temperature measurements and the control of the H-bridge are both performed by the on board computer.

A method of detecting an airborne substance may include moving an air sample through an air flow channel of a biochip. The airborne substance in the air sample diffuses through a membrane in the biochip separating the air flow channel from a plurality of wells holding living cells. The living cells react to exposure to the airborne substance, such as a VOC and emit fluorescent light. The fluorescent light is detected indicating the presence of the airborne substance in the air sample. The cells may be illuminated by a light source.

Thus systems and methods have been shown and described. Various changes and substitutions may of course be made without departing from the spirit and scope of the invention. The invention, therefore, should not be limited, except by the following claims and their equivalents. Application Nos. 63/189,015; Ser. Nos. 17/153,747; 16/486,132; 16/344,791; and 17/251,557 are incorporated herein by reference.

1. A biochip, comprising:
one or more wells containing genetically modified living cells expressing an odorant receptor capable of binding to a volatile organic compound and a fluorescent reporter that fluoresces in response to binding of the volatile organic compound to the odorant receptor;
a capillary connecting each well to a liquid source;
an air flow channel separated from each well by a membrane, the air flow channel having an air inlet and an air outlet, wherein the living cells are bound to a first side of the membrane, and a wall of the air flow channel is formed by a second side of the membrane; and
wherein at least a portion of the biochip is transparent.

2. The biochip of claim 1 comprising a plurality of flat transparent layers attached together.

3. The biochip of claim 1 wherein the liquid source comprises one or more liquid containing reservoirs in the biochip connected to one or more of the wells by a capillary.

4. The biochip of claim 1 comprising a plurality of wells with each well connected by a capillary to a single liquid reservoir in the biochip.

5. The biochip of claim 1 wherein the air flow channel extends under each of the wells in an S-shaped configuration.

6. The biochip of claim 1 wherein the membrane is treated to make it transparent and/or to promote cell adhesion.

7. The biochip of claim 1 wherein the membrane encloses the air flow channel from above and a first layer of the biochip encloses the air flow channel from below, the liquid source comprises a liquid inlet in the biochip and the liquid inlet, the air inlet and the air outlet are on a first surface of the biochip and are sealed by a pierceable seal layer.

8. The biochip of claim 1 wherein the air flow channel is wider at positions under the wells than at other positions.

9. The biochip of claim 1 having multiple layers wherein the membrane is on the bottom surface of a layer and closes off the bottom of the wells.

10. The biochip of claim 1 wherein the cells express one unique odorant receptor.

11. The biochip of claim 1 wherein the cells express more than one unique odorant receptor.

12. The biochip of claim 1 wherein the fluorescent reporter comprises a calcium sensitive fluorescent reporter.

13. A system for detecting VOC's in air, comprising:
an optical system in a housing, the optical system adapted to detect fluorescence;
an electronic system electrically connected to one or more light sources and light detectors in the housing;
a frame in the housing, the frame forming a biochip slot;
a top plate vertically movable towards and away from the frame;
an air inlet and air outlet in the top plate; and
a pump in the housing connected to the air outlet.

14. The system of claim 13 further comprising a biochip having one or more wells containing genetically modified living cells expressing an odorant receptor capable of binding to a volatile organic compound and a fluorescent reporter that fluoresces in response to binding the volatile organic compound to the odorant receptor; a capillary connecting each well to a liquid source; an air flow channel separated from each well by a membrane, the air flow channel having an air inlet and an air outlet, wherein the living cells are on bound to a first side of the membrane, and a second side of the membrane forms a surface of the air flow channel; wherein at least a portion of the biochip is transparent; and the air inlet and the air outlet in the top plate are aligned with the air inlet and the air outlet of the air flow channel.

15. The system of claim 14 wherein the optical system has multiple light sources and multiple light detectors, with one light source and one light detector aligned with each of the wells.

16. The system of claim 15 further including:
a loader including:
a lift plate vertically movable in a frame;
an elevator for lifting and lowering the lift plate;
a guideway within the frame, the guideway having a loader slot; and
a biochip mover for moving the biochip from the guideway into a detection system slot.

17. A method of detecting an airborne substance, comprising:
moving an air sample through an air flow channel of a biochip;
the airborne substance in the air sample diffusing through a membrane in the biochip separating the air flow channel from a plurality of wells holding genetically modified living cells;
the living cells expressing one or more odorant receptor capable of binding to the airborne substance and a fluorescent reporter that fluoresces in response to binding of the substance to the odorant receptor; and
detecting fluorescent light emitted from the living cells.

18. The method of claim 17 further including illuminating the living cells with light.

19. The method of claim 17 further including analyzing detected fluorescent light to translate it into a detection event indicating detection of an airborne substance.

20. The method of claim 17 wherein the living cells are bound to a first side of the membrane, and a second side of the membrane forms a surface of the air flow channel, and wherein at least a portion of the biochip is transparent.
